(19) United States
(12) Patent Application Publication
(10) Pub. No.: US 2025/0288412 A1
(43) Pub. Date: Sep. 18, 2025

(54) CORONARY PERFUSION DEVICE

(71) Applicant: EDWARDS LIFESCIENCES CORPORATION, Irvine, CA (US)

(72) Inventor: Noam Miller, Givatayim (IL)

(73) Assignee: EDWARDS LIFESCIENCES CORPORATION, Irvine, CA (US)

(21) Appl. No.: 19/225,740

(22) Filed: Jun. 2, 2025

Related U.S. Application Data
(63) Continuation of application No. PCT/US2023/080473, filed on Nov. 20, 2023.
(60) Provisional application No. 63/432,329, filed on Dec. 13, 2022.

(51) Int. Cl. A61F 2/24

(52) U.S. Cl. CPC A61F 2/2409; A61F 2250/00

(57) ABSTRACT

A coronary perfusion device comprises a first conduit and a second conduit. The first conduit and the second conduit can extend through the coronary ostia into the left and right coronary arteries when the coronary perfusion device is installed downstream of the aortic valve. In some examples, the coronary perfusion device comprises a circumferential channel extending between the first conduit and the second conduit. The circumferential channel can also define a trough to collect retrograde blood flow. The coronary perfusion device can also comprise an annular frame to secure the first conduit, the second conduit, and the circumferential channel relative to the native aorta. In some examples, the annular frame can be radially expandable or compressible to facilitate transcatheter delivery.

### CROSS REFERENCE TO RELATED APPLICATIONS

This application is a continuation of PCT Application No. PCT/US2023/080473, filed on Nov. 20, 2023, which claims the benefit of U.S. Provisional Application No. 63/432,329, filed Dec. 13, 2022, each of these applications being incorporated by reference herein in its entirety.

### FIELD

The present disclosure relates to coronary perfusion devices and methods of implanting the same.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size. The prosthetic heart valve can include valve structure (for example, leaflets) for regulating blood flow in a single direction.

During the replacement of an aortic valve, a prosthetic aortic valve is conventionally implanted in the native aortic annulus, and extends from the native aortic annulus along the aortic root. A potential risk associated with implanting a prosthetic valve within the native aortic annulus is that the native leaflets, situated between the frame of the prosthetic valve and the aortic vessel wall, may be pressed closer to or against the coronary ostia, thereby interfering with coronary blood perfusion. The risk of interference with coronary blood perfusion is heightened with valve-in-valve (ViV) procedures, in which a replacement prosthetic valve (referred to as a guest valve) is implanted within a previously implanted prosthetic valve (referred to as a host valve), for replacing the function of the host valve. In such cases, the guest valve, which is typically positioned higher than (that is, downstream of) the native annulus, may press the leaflets of the host valve toward the coronary ostia, resulting in similar interference with blood flow into the coronaries.

There is, therefore, a need to provide devices that will ensure adequate perfusion to the coronary arteries, even if native leaflets or leaflets of prosthetic valves are urged against the coronary ostia.

### SUMMARY

A coronary perfusion device can comprise a conduit configured to extend into a coronary artery and direct a retrograde flow of blood into the coronary artery.

In some examples, the coronary perfusion device can comprise a first conduit and a second conduit, the first conduit can extend into a left coronary artery, and the second conduit can extend into a right coronary artery.

In some examples, a coronary perfusion device can comprise a trough extending between the first conduit and the second conduit.

In some examples, the trough of the coronary perfusion device can have a tapered cross-sectional profile.

In some examples, the trough of the coronary perfusion device comprises a flexible material.

In some examples, the trough of the coronary perfusion device comprises a mouth configured to open when the heart of the patient is in diastole, and close or partially close when the heart is in systole.

In some examples, the coronary perfusion device comprises an annular frame, wherein the first conduit and the second conduit are attached to the annular frame.

In some examples, the coronary perfusion device comprises a skirt extending around an outer surface of the frame.

In some examples, the annular frame of the coronary perfusion device is radially expandable and compressible.

Certain examples concern a coronary perfusion device for implantation in an ascending aorta of a patient. The coronary perfusion device comprises at least one conduit and a circumferentially extending trough comprising an opening. The conduit is connected to the trough at the opening, the device is configured to be implanted in the ascending aorta such that the conduit extends into a coronary artery, and the trough is configured to receive retrograde blood and direct the retrograde blood to flow through the conduit into the coronary artery.

Certain examples concern a coronary perfusion device for implantation in an ascending aorta of a patient. The coronary perfusion device comprises an annular frame and a trough at least partially disposed inside the frame. The trough comprises an inner wall, an outer wall, an end wall extending between the inner wall and the outer wall, a first opening, and a second opening. The coronary perfusion device also comprises a first conduit extending from the first opening, a second conduit extending from the second opening, and a skirt extending around an outer surface of the frame. The device is configured to be implanted in the ascending aorta such that the first conduit extends into a right coronary artery and the second conduit extends into a left coronary artery. The trough is configured to receive retrograde blood and direct the retrograde blood flow through the first and second conduits into the right coronary artery and the second coronary artery, respectively.

Certain examples concern a method for implanting a coronary perfusion device, the coronary perfusion device including a first conduit, a second conduit, and a circumferential trough connecting the first conduit and the second conduit. The method comprises implanting the trough in the ascending aorta of a patient downstream of the right and left coronary arteries, positioning the first conduit in the right coronary artery of the patient, and positioning the second conduit in the left coronary artery of the patient.

The above method(s) can be performed on a living animal or on a simulation, such as on a cadaver, cadaver heart, anthropomorphic ghost, simulator (for example, with body parts, heart, tissue, etc. being simulated).

In some examples, the coronary profusion device comprises one or more of the components recited in examples 1-21 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a coronary perfusion device implanted within an ascending aorta of a patient, according to one example.

FIG. 2 is a perspective view of the coronary perfusion device of FIG. 1.

FIG. 3A is a cutaway view of the coronary perfusion device of FIG. 1 and the aorta.

FIG. 3B is a top elevation view of the coronary perfusion device of FIG. 1

FIG. 4A is a side view of a coronary perfusion device implanted within an ascending aorta of a patient, according to another example.

FIG. 4B is a top elevation view of the coronary perfusion device of FIG. 4A.

FIG. 4C is a cutaway view of the coronary perfusion device of FIG. 4A.

FIG. 5 is a top elevation view of a coronary perfusion device according to another example.

### DETAILED DESCRIPTION

### General Considerations

For purposes of this description, certain aspects, advantages, and novel features of the examples of this disclosure are described herein. The described methods, systems, and apparatus should not be construed as limiting in any way. Instead, the present disclosure is directed toward all novel and non-obvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Introduction to the Disclosed Technology

The muscles of the human heart require a flow of blood, which is supplied by the coronary arteries (that is, the left coronary artery and the right coronary artery). In a healthy heart, this flow of blood is provided by the perfusion of the coronary arteries during the diastolic phase of the heartbeat.

When a native aortic valve is replaced by a prosthetic heart valve, the leaflets of the native aortic valve can be pressed outwardly toward the coronary ostia. In some cases, this can reduce or impair perfusion of the coronary arteries. A valve-in-valve (ViV) procedure, in which a second prosthetic valve (guest valve) is implanted within a first prosthetic heart valve (host valve), may increase the risk of reduced perfusion because the leaflets of the host valve may also be pressed outwardly towards the coronary arteries.

Coronary perfusion devices, such as the coronary perfusion devices disclosed herein, direct retrograde blood flow into one or both of the left coronary artery and the right coronary artery. When such devices are implanted in a patient, one or more conduits can extend through the coronary ostia and into the coronary arteries. During diastole, retrograde blood can flow into the one or more conduits and thus into the left coronary artery and/or the right coronary artery. Thus, the coronary arteries may be perfused through the one or more conduits even if the native and/or prosthetic leaflets are urged against the coronary ostia.

Coronary perfusion devices disclosed herein can be radially compressible and expandable between a radially compressed state and a radially expanded state. Thus, the coronary perfusion devices can be crimped on or retained by an implant delivery apparatus in the radially compressed state during delivery, and then expanded to the radially expanded state once the coronary perfusion device reaches the implantation site. It is understood that the coronary perfusion devices disclosed herein may be used with a variety of implant delivery apparatuses and can be implanted via various delivery procedures, examples of which will be discussed in more detail later.

In some examples, the coronary perfusion devices disclosed herein can be implanted along with a prosthetic heart valve, such as disclosed in U.S. Pat. No. 9,393,110, which is incorporated by reference herein.

### Examples of the Disclosed Technology

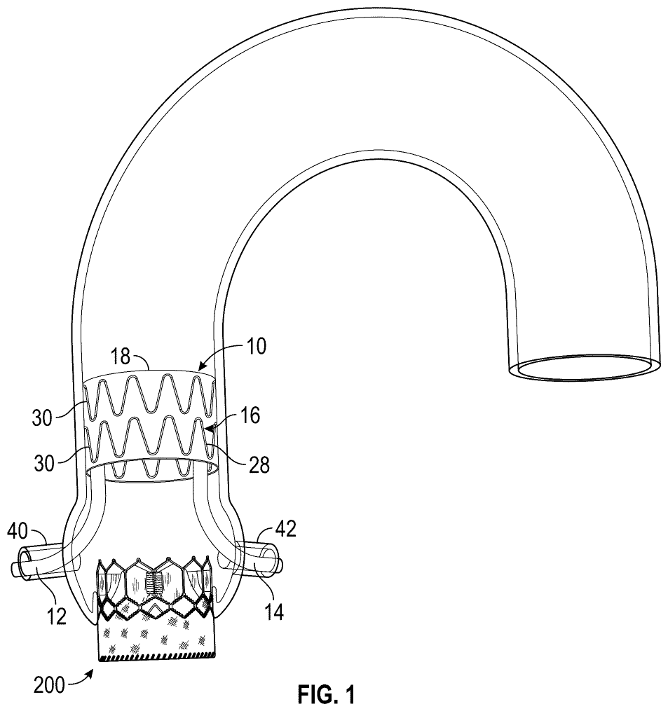

FIG. 1 shows an example coronary perfusion device 10. The coronary perfusion device 10 can be implanted in the ascending aorta of a patient, downstream of the native aortic valve and the coronary arteries to facilitate coronary perfusion of retrograde blood flow. The coronary perfusion device 10 can comprise a first conduit 12, a second conduit 14, and an annular frame 16. The first conduit 12 and the second conduit 14 can be coupled to an inner surface of the annular frame 16, and can extend upstream (that is, towards the coronary arteries) from the annular frame 16.

The annular frame 16, in some examples, can be radially expandable, and can be moved between a radially compressed state and a radially expandable state. For example, the annular frame 16 can be advanced through the vasculature of a patient to a desired implantation site in the radially compressed state, and then radially expanded until the annular frame 16 engages the native vasculature of the patient's aorta.

In some examples, the coronary perfusion device 10 is surgically implanted (for example, during open heart surgery). In such examples, the annular frame 16 need not be radially expandable and compressible, and may in some examples be a rigid frame of fixed diameter.

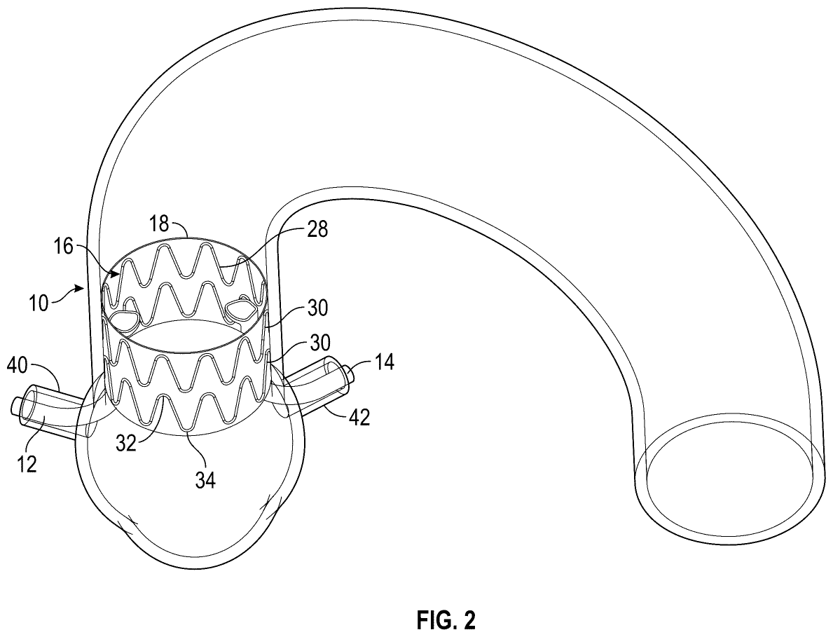

The annular frame 16 can comprise a plurality of interconnected struts 28. In some examples, such as that shown in FIG. 2, the interconnected struts 28 can be arranged into one or more circumferential rings 30 (for example two rings 30, as shown in the example of FIG. 2). Each circumferential ring 30 can comprise a plurality of peaks 32 and valleys 34. While the example shown in FIG. 2 comprises an annular frame 16 having two circumferential rings 30, it is to be understood that some examples can comprise an annular frame 16 having one circumferential ring 30, or more than two circumferential rings 30, such as three, four, five, or six circumferential rings 30, depending on the desired length of the annular frame 16.

The frame 16 can be made of any of various suitable plastically-expandable materials (for example, stainless steel, etc.) or self-expanding materials (for example, Nitinol) as known in the art. When constructed of a plastically-expandable material, the frame 16 (and thus the perfusion device 10) can be crimped to a radially compressed state on a delivery catheter and then expanded inside a patient by an inflatable balloon or equivalent expansion mechanism. When constructed of a self-expandable material, the frame 16 (and thus the perfusion device 10) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 16) include, metal alloys, polymers, or combinations thereof. Example metal alloys can comprise one or more of the following: nickel, cobalt, chromium, molybdenum, titanium, or other biocompatible metal. In some examples, the frame 16 can comprise stainless steel. In some examples, the frame 16 can comprise cobalt-chromium. In some examples, the frame 16 can comprise nickel-cobalt-chromium. In some examples, the frame 16 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

With continued reference to FIG. 2, the coronary perfusion device 10 can also include a skirt 18 (sometimes called a sealing element 18) extending around an outer surface of the annular frame 16. In such examples, the skirt 18 is disposed between the interior of the native ascending aorta and the annular frame 16, as shown in FIG. 2. The skirt 18 can form a seal against the interior of the native ascending aorta, to prevent leakage or reduce the flow of blood around the exterior of the annular frame 16. The seal between the interior of the native ascending aorta and the skirt 18 can be generated by the expansion of the frame 16 of the perfusion device 10. As the frame 16 expands, for example, from a crimped or radially compressed state to a functional or radially expanded state, the skirt 18 can be pressed against the interior of the native ascending aorta to generate the seal. In some examples, the seal between the interior of the native ascending aorta and the skirt 18 can prevent or reduce the likelihood of the perfusion device 10 from slipping downstream (for example, downstream and away from the coronary arteries 40, 42) and therefore help with maintaining the position of the perfusion device 10 with respect to the coronary arteries.

The frame 16, and in particular the rings 30, can be connected to the skirt 18 with sutures (not shown). In some examples, the skirt 18 can be mounted to the inner surface of the frame 16 (that is, the frame 16 extends around the outer surface of the skirt 18). In some examples, the perfusion device 10 can include a skirt 18 extending around the outer surface of the frame 16 and an additional skirt (not shown) mounted to the inner surface of the frame 16. A skirt mounted to the outer surface of the frame 16 can be referred to as an outer skirt and a skirt mounted to the inner surface of the frame 16 can be referred to as an inner skirt.

The skirt 18 can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plush nap or pile include velour, velvet, velveteen, corduroy, terrycloth, fleece, etc. In some examples, the skirt can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources).

As shown in FIGS. 1-3B, the first conduit 12 and the second conduit 14 are sized to extend into the coronary arteries of the heart of the patient. For example, as shown in FIG. 2, the first conduit 12 can extend into the right coronary artery 40, and the second conduit 14 can extend into the left coronary artery 42. In this way, retrograde blood flow in the ascending aorta (for example, blood flowing in a direction from the perfusion device 10 towards the coronary arteries during diastole) can flow into and through the conduits 12, 14 and into both the left coronary artery and the right coronary artery to provide blood flow to the heart muscles.

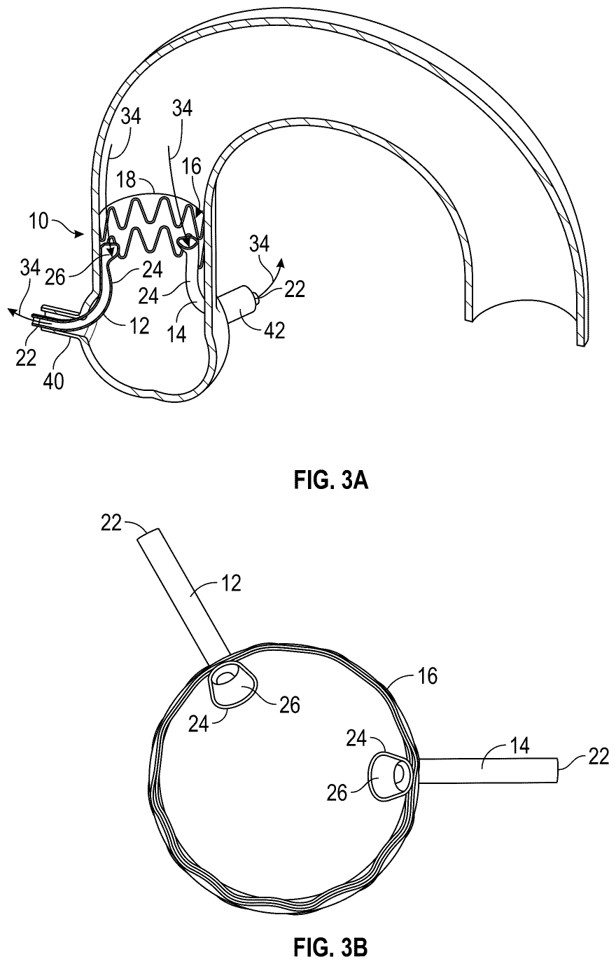

Turning now to FIG. 3A, the conduits 12, 14, can each comprise a first end portion 22 (an outflow end portion) and a second end portion 24 (an inflow end portion). The first end portion 22 can extend away from the annular frame 16 and into a corresponding coronary artery of the patient (that is, the first end portion 22 can extend upstream of the annular frame 16, in the direction of the retrograde flow). The second end portion 24 of each conduit 12, 14 can be disposed within and attached to the annular frame 16 and/or the skirt 18, allowing the annular frame 16 to secure the position of the first conduit 12 and the second conduit 14 relative to the aorta of the patient. The second end portions 24 of the conduits 12, 14 can be attached to the frame 16 and/or the skirt 18, such as with sutures, an adhesive, by welding, mechanical fasteners (for example, clamps), and/or various other connection means.

As shown in FIG. 3B, the second end portion 24 of each of the conduits 12, 14 can have an enlarged or funnel-shaped mouth 26. The mouth 26 can increase the volume of blood from the retrograde blood flow that is captured by the conduits 12, 14, thus increasing the supply of blood to the coronary arteries of the patient. During diastole, retrograde blood can flow into the open ends of the second end portions 24 of the conduits, through the conduits, and outwardly through the open ends of the first end portions 22 into the coronary arteries 40, 42, in the directions indicated by arrows 34.

In some examples, the conduits 12, 14, or particularly the second end portion 24 of each end conduit, can be comprised of a flexible material. In this way, antegrade blood flow (that is, blood flow from the aortic valve into the aorta when the heart is in systole) can push radially outwards against the second end portion 24 of each of the conduits 12, 14. This flattens the mouth 26 of each conduit 12, 14 against the frame, minimizing the profile of the conduits 12, 14 which could impede the antegrade blood flow. However, when blood is flowing in the retrograde direction (that is, when the heart is in diastole), the retrograde blood flow can press against the inner surface of the second end portion 24 of each conduit 12, 14. This can urge the mouth 26 of the conduits 12, 14 open, increasing the portion of the retrograde blood flow captured by the conduits 12, 14 and directed into the coronary arteries of the patient.

In some examples, the conduits 12, 14 can be made from any of materials disclosed above for forming the skirt 18. In some examples, the conduits 12, 14 can be made from any suitably flexible and biocompatible elastomer or polymer material, such as silicone, latex, nylon, polyester, or a combination thereof.

Returning to FIG. 1, the coronary perfusion device 10 can be implanted at the same time that a prosthetic heart valve 200 is implanted in the native aortic valve, or after a prosthetic heart valve 200 has been implanted in a subsequent procedure. In some examples, the prosthetic heart valve 200 can be implanted in the native aortic valve of the patient, upstream of the coronary perfusion device 10 as shown in FIG. 1. While the prosthetic heart valve 200 is not shown in FIGS. 2-3B, it is to be understood that the prosthetic heart valve 200 can be present upstream of the coronary perfusion device 10 as illustrated in these figures.

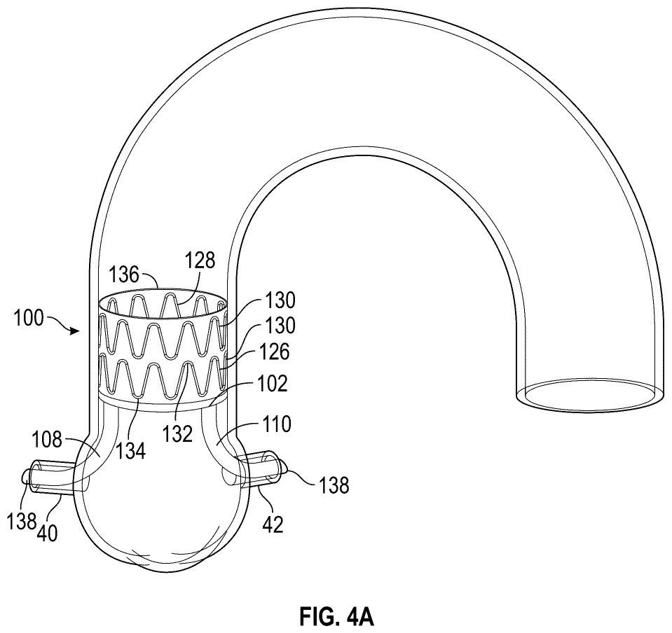

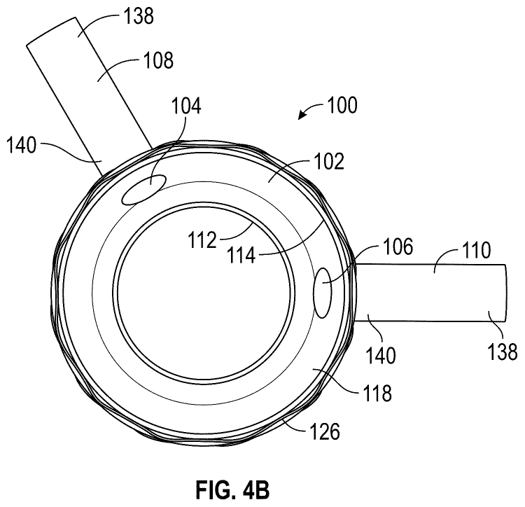

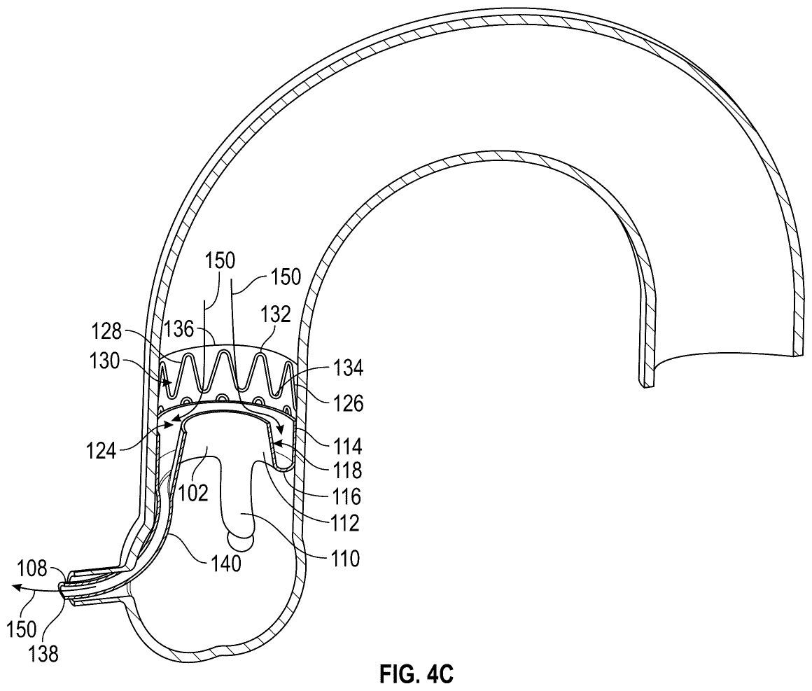

FIGS. 4A-4C show an example coronary perfusion device 100. The coronary perfusion device 100 can be implanted in the ascending aorta of a patient downstream of the native aortic valve and the coronary arteries to facilitate coronary perfusion during diastole of the heart, and functions in a fashion similar to the coronary perfusion device 10, except for the differences disclosed below.

As shown in FIG. 4B, the coronary perfusion device 100 can comprise a trough 118 defining a circumferential channel 102. The trough 118 comprises a first opening 104 and a second opening 106, both of which are in fluid communication with the channel 102. The coronary perfusion device 100 also comprises a first conduit 108 extending from the first opening 104 and a second conduit 110 extending from the second opening 106.

Referring to FIGS. 4B and 4C, the trough 118 can also comprise an inner wall 112 and an outer wall 114. An end wall, or bottom wall, 116 can extend between the inner wall 112 and the outer wall 114. The trough 118 can be open in the downstream direction (that is, away from the aortic valve), so that when blood is in retrograde flow in the aorta of the patient, a portion of the retrograde flow of blood is captured by the trough 118. The openings 104, 106 desirably are formed in the end wall 116 as shown. In some examples, the openings 104, 106 can be at least partially or entirely formed in one of the inner wall 112 and the outer wall 114.

In some examples, the circumferential channel 102 can have a tapered cross-sectional profile or wedge-shaped cross section. As shown in FIG. 4C, the outer wall 114 can be vertical or substantially vertical, such that it extends parallel or substantially parallel to the ascending aorta of the patient. The inner wall 112 can extend radially inwards along its length in the direction of antegrade flow (that is, away from the aortic valve). In this way, the width of the trough 118 can be greatest at the portion of the circumferential channel 102 furthest away from the aortic valve, allowing the circumferential channel 102 to capture a relatively greater portion of the retrograde flow of blood.

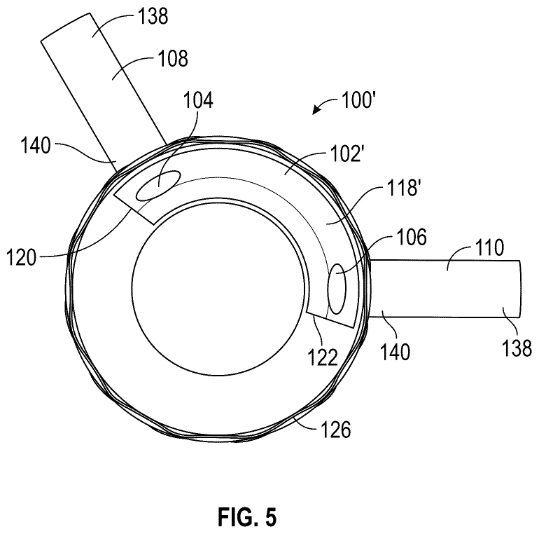

In some examples, such as that illustrated in FIG. 4B, the trough 118 can be an annular trough and the circumferential channel 102 can be a circular channel or an annular channel that extends a full 360-degrees around a central longitudinal axis of the device 100. In some examples, such as that illustrated in FIG. 5, a perfusion device 100′ includes a partial annular trough 118′ that defines a circumferential channel 102′ circumscribing an arc length around a central longitudinal axis of the device that is less than 360 degrees. For example, the channel 102′ can comprise a portion of a circle. In such examples, the trough 118 can have a first side wall 120 and a second side wall 122. The first opening 104 can be positioned proximate the first side wall 120 and the second opening 106 can be positioned proximate the second side wall 122.

In some examples, the circumferential channel 102 or the circumferential channel 102′ can comprise a flexible material that allows the inner wall 112 to deflect radially outwards or inwards, relative to the outer wall 114 under the flow of blood. For example, when blood is moving in the antegrade direction (for example, when the heart is in systole), the flow of blood can tend to urge the inner wall 112 to deflect towards the outer wall 114, closing or partially closing a mouth 124 of the trough 118, thereby reducing any restriction to the antegrade flow of blood through the coronary perfusion device 100. When blood is moving in the retrograde direction (for example, when the heart is in diastole), the flow of blood can urge the inner wall 112 to deflect away from the outer wall 114, opening or further opening the mouth 124 of the trough. This increases the cross-sectional area of the circumferential channel 102 at the mouth 124, and thus increases the volume of blood captured in the circumferential channel 102 when blood is flowing in the retrograde direction. The trough 118 or trough 118′ can be made from any of the materials disclosed above for forming the skirt 18. In some examples, the trough 118 or trough 118′ can include reinforcing struts (for example, metal struts) embedded in or attached to the inner wall 112, the outer wall 114, and/or the end wall 116. Flexible materials suitable making the trough 118, 118′ include any suitably flexible and biocompatible elastomer or polymer material, such as silicone, latex, polyester, nylon, or a combination thereof.

Advantageously, a coronary profusion device such as coronary profusion device 100 (or device 100′) having the circumferential channel 102 (or the circumferential channel 102′) extending between the first conduit 108 and the second conduit 110 may provide a larger inlet volume than a coronary profusion device omitting the circumferential channel 102 (or the circumferential channel 102′), and thus such example coronary perfusion devices can supply a greater quantity of blood to the coronary arteries.

The coronary perfusion device 100 illustrated in FIGS. 4A-4C and the device 100′ of FIG. 5 can also comprise an annular frame 126. The annular frame 126 can be positioned radially outwards of the trough 118. In some examples, the annular frame 126 can be radially expandable and radially compressible, to enable transcatheter delivery and deployment of the coronary perfusion device 100. The frame 126 can be plastically expandable or self-expandable and can be made from any of the materials disclosed above in connection with the frame 16. In some examples, the coronary perfusion device 100 may be intended for surgical implantation, such as during an open-heart surgical procedure. In such examples, the annular frame 126 need not be radially expandable or radially compressible, and an annular frame 126 with greater rigidity may be used instead.

In some examples, the annular frame 126 comprises a plurality of interconnected struts 128. In some examples, such as that shown in FIG. 4A, the interconnected struts 128 can be arranged into one or more circumferential rings 130. Each circumferential ring 130 can comprise a plurality of peaks 132 and valleys 134. While the example shown in FIG. 4A comprises an annular frame 126 having two circumferential rings 130, it is to be understood that some examples can comprise an annular frame 126 having one circumferential ring 130, or more than two circumferential rings 130, such as three, four, five, or six circumferential rings 130, depending on the desired length of the annular frame 126.

With continued reference to FIG. 4A, the coronary perfusion device 100 can also comprise a skirt or sealing member 136 (similar to skirt 18) disposed around the external surface of the annular frame 126. The skirt 136 is thus positioned between the annular frame 126 and the native vasculature of the patient when the coronary perfusion device 100 is implanted in the patient. This can prevent leakage or reduce the flow of blood around the annular frame 126. The skirt 136 can be made from any of the materials disclosed above for the skirt 18 and can be coupled to the frame 126 in the same manner as disclosed above for coupling the skirt 18 to the frame 16. Moreover, the device 100 (or the device 100′) can include a skirt positioned on the inner surface of the frame 126, or on the external surface and the inner surface of the frame 126. The seal between the interior of the native ascending aorta and the skirt 136 can be generated by the expansion of the frame 126 of the perfusion device 100. As the frame 126 expands, for example, from a crimped or radially compressed state to a functional or radially expanded state, the skirt 136 can be pressed against the interior of the native ascending aorta to generate the seal. In some examples, the seal between the interior of the native ascending aorta and the skirt 136 can prevent or reduce the likelihood of the perfusion device 100 (or the device 100′) from slipping downstream (for example, downstream and away from the coronary arteries 40, 42) and therefore help with maintaining the position of the perfusion device 100 with respect to the coronary arteries.

The trough 118 can be attached to the interior surface of the annular frame 126 and/or the skirt 136, such as by securing the outer wall 114 the annular frame 126 and/or the skirt 136, such as with sutures, an adhesive, by welding, mechanical fasteners (for example, clamps), and/or various other connection means. Thus, when the annular frame 126 is engaged with the native vasculature of the patient, the location of the trough 118 and the conduits 108, 110 can be fixed relative to the ascending aorta and aortic valve of the patient.

In some examples, the devices 100, 100′ can omit the frame 126 and the skirt 136. In such examples, the devices 100, 100′ can be implanted by suturing or otherwise securing the outer wall 114 of the trough 118, 118′ directly to the inner wall of the aorta.

The conduits 108, 110 can each have a first end portion 138 (an outflow end portion) and a second end portion 140 (an inflow end portion). The conduits 108, 110 can be attached at their second end portions 140 to the trough 118 at the first opening 104 and the second opening 106 respectively. As described herein regarding the coronary perfusion device 10, and illustrated in FIG. 4A, the first end portion 138 of the first conduit 108 can extend into the right coronary artery 40, and the first end portion of the second conduit 110 can extend into the left coronary artery 42 of the patient.

While FIGS. 4A-5 do not show a prosthetic heart valve, such as the prosthetic heart valve 200 (as shown in FIG. 1 and described herein), it is to be understood that the coronary perfusion device 100 can be implanted downstream of such a prosthetic heart valve in some examples. In such examples, the prosthetic heart valve 200 can be implanted in the native aortic heart valve, or in a host valve implanted in the native aortic heart valve (for example, as in a valve-in-valve procedure). The coronary perfusion device 100 can be implanted along with the prosthetic heart valve 200 during the same procedure, or in a subsequent procedure after the prosthetic heart valve 200 has already been implanted.

Once implanted, during diastole, retrograde blood can flow through the open mouth 124 and into the trough 118, where the blood is directed to flow through the openings 104, 106, the conduits 108, 110, and outwardly through the open ends of the first end portions 138 into the coronary arteries 40, 42, in the directions indicated by arrows 150.

The coronary perfusion devices 10, 100, 100′ described herein can direct retrograde blood flow into the coronary arteries of the patient. This may be particularly advantageous for patients with one or more implanted prosthetic heart valves, where native or prosthetic valve leaflets are urged against or in close proximity to the coronary ostia, causing a reduction in the flow of blood into the coronary arteries.

It is to be appreciated that while the coronary perfusion devices 10, 100, 100′ disclosed herein are illustrated and described as comprising first and second conduits, in some examples the coronary perfusion devices 10, 100 can comprise a single conduit extending into either the left coronary artery or the right coronary artery.

### Delivery Techniques

For implanting a coronary perfusion device in the ascending aorta via a transfemoral delivery approach, the coronary perfusion device can be mounted in a radially compressed state along the distal end portion of a delivery apparatus. The coronary perfusion device and the distal end portion of the delivery apparatus are inserted into a femoral artery and are advanced into and through the descending aorta, around the aortic arch, and through the ascending aorta. The coronary perfusion device is positioned in the native ascending aorta and radially expanded (for example, by inflating a balloon, actuating one or more actuators of the delivery apparatus, or deploying the prosthetic valve from a delivery capsule to allow the coronary perfusion device to self-expand). Alternatively, a coronary perfusion device can be implanted within the native ascending aorta in a transapical procedure, whereby the coronary perfusion device (on the distal end portion of the delivery apparatus) is introduced into the left ventricle through a surgical opening in the chest and the apex of the heart, and the coronary perfusion device is positioned within the native ascending aorta. Alternatively, in a transaortic procedure, a coronary perfusion device (on the distal end portion of the delivery apparatus) is introduced into the aorta through a surgical incision in the ascending aorta, such as through a partial J-sternotomy or right parasternal mini-thoracotomy, and then advanced through the ascending aorta toward the desired implantation location.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc., as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A coronary perfusion device for implantation in an ascending aorta of a patient, comprising: at least one conduit; and a circumferentially extending trough comprising an opening; wherein the conduit is connected to the trough at the opening; wherein the device is configured to be implanted in the ascending aorta such that the conduit extends into a coronary artery; and wherein the trough is configured to receive retrograde blood and direct the retrograde blood to flow through the conduit into the coronary artery.

Example 2. The coronary perfusion device of any example herein, particularly example 1, wherein: the at least one conduit comprises first and second conduits; the opening in the trough comprises a first opening and a second opening; the first conduit is connected to the trough at the first opening and has an outflow end portion configured to be inserted into the right coronary artery; and the second conduit is connected to the trough at the second opening and has an outflow end portion configured to be inserted into the left coronary artery.

Example 3. The coronary perfusion device of any example herein, particularly any of examples 1-2, wherein the trough comprises an inner wall, an outer wall, and an end wall connecting the inner wall and the outer wall to define a channel.

Example 4. The coronary perfusion device of any example herein, particularly example 3 when dependent on example 2, wherein the first opening and the second opening are formed in the end wall at circumferentially spaced locations.

Example 5. The coronary perfusion device of any example herein, particularly any of examples 3-4, wherein the inner wall is angularly movable toward and away from the outer wall under the flow of blood.

Example 6. The coronary perfusion device of any example herein, particularly any of examples 1-5, wherein the coronary perfusion device comprises woven polyester, silicone, latex, nylon, or any combination thereof.

Example 7. The coronary perfusion device of any example herein, particularly any of examples 1-6, wherein the trough is an annular trough or a partial annular trough.

Example 8. The coronary perfusion device of any example herein, particularly any of examples 1-7, wherein the coronary perfusion device is radially expandable and compressible.

Example 9. The coronary perfusion device of any example herein, particularly any of examples 1-8, further comprising an annular frame, and wherein the trough is disposed at least partially within the frame.

Example 10. The coronary perfusion device of any example herein, particularly example 9, further comprising a skirt extending around an outer surface of the frame.

Example 11. A coronary perfusion device for implantation in an ascending aorta of a patient comprising: an annular frame; a trough at least partially disposed inside the frame, the trough comprising an inner wall, an outer wall, an end wall extending between the inner wall and the outer wall, a first opening, and a second opening; a first conduit extending from the first opening; a second conduit extending from the second opening; and a skirt extending around an outer surface of the frame; wherein the device is configured to be implanted in the ascending aorta such that the first conduit extends into a right coronary artery and the second conduit extends into a left coronary artery; and wherein the trough is configured to receive retrograde blood and direct the retrograde blood flow through the first and second conduits into the right coronary artery and the second coronary artery, respectively.

Example 12. The coronary perfusion device of any example herein, particularly example 11, wherein the frame and the trough are radially expandable and compressible between a radially expanded state and a radially compressed state.

Example 13. The coronary perfusion device of any example herein, particularly any of examples 11-12, wherein the skirt is configured to engage the native ascending aorta to form a seal between the coronary perfusion device and the native ascending aorta.

Example 14. The coronary perfusion device of any example herein, particularly any of examples 11-13, wherein the trough comprises an open mouth defined between the inner wall and the outer wall for receiving retrograde blood.

Example 15. The coronary perfusion device of any example herein, particularly example 14, wherein the mouth is configured to close or partially close when blood flows through the ascending aorta in an antegrade direction.

Example 16. The coronary perfusion device of any example herein, particularly any of examples 14-15, wherein the mouth is configured to open or further open when blood flows through the ascending aorta in a retrograde direction.

Example 17. The coronary perfusion device of any example herein, particularly example 16, wherein the trough has a tapered cross-sectional profile when blood flows through the ascending aorta in a retrograde direction.

Example 18. The coronary perfusion device of any example herein, particularly any of examples 11-17, wherein the trough is annular or partially annular.

Example 19. The coronary perfusion device of any example herein, particularly any of examples 11-18, wherein the first opening and the second opening are located in the end wall.

Example 20. A method for implanting a coronary perfusion device, the coronary perfusion device including a first conduit, a second conduit, and a circumferential trough connecting the first conduit and the second conduit, comprising: implanting the trough in the ascending aorta of a patient downstream of the right and left coronary arteries; positioning the first conduit in the right coronary artery of the patient; and positioning the second conduit in the left coronary artery of the patient.

Example 21. A coronary perfusion device of any example herein, particularly any of examples 1-20, wherein the coronary perfusion device is sterilized.

The features described herein with regard to any example can be combined with other features described in any one or more of the other examples, unless otherwise stated. For example, any one or more of the features of one coronary perfusion device can be combined with any one or more features of another coronary perfusion device.

In view of the many possible embodiments to which the principles of the disclosure may be applied, it should be recognized that the illustrated configurations depict examples of the disclosed technology and should not be taken as limiting the scope of the disclosure or the claims. Rather, the scope of the claimed subject matter is defined by the following claims and their equivalents.

1. A coronary perfusion device for implantation in an ascending aorta of a patient, comprising:
at least one conduit; and
a circumferentially extending trough comprising an opening;
wherein the conduit is connected to the trough at the opening;
wherein the device is configured to be implanted in the ascending aorta such that the conduit extends into a coronary artery; and
wherein the trough is configured to receive retrograde blood and direct the retrograde blood to flow through the conduit into the coronary artery.

2. The coronary perfusion device of claim 1, wherein:
the at least one conduit comprises first and second conduits;
the opening in the trough comprises a first opening and a second opening;
the first conduit is connected to the trough at the first opening and has an outflow end portion configured to be inserted into the right coronary artery; and
the second conduit is connected to the trough at the second opening and has an outflow end portion configured to be inserted into the left coronary artery.

3. The coronary perfusion device of claim 2, wherein the trough comprises an inner wall, an outer wall, and an end wall connecting the inner wall and the outer wall to define a channel, and wherein the first opening and the second opening are formed in the end wall at circumferentially spaced locations.

4. The coronary perfusion device of claim 1, wherein the trough comprises an inner wall, an outer wall, and an end wall connecting the inner wall and the outer wall to define a channel, wherein the opening is formed in the end wall.

5. The coronary perfusion device of claim 4, wherein the inner wall is angularly movable toward and away from the outer wall under the flow of blood.

6. The coronary perfusion device of claim 1, wherein the coronary perfusion device comprises woven polyester, silicone, latex, nylon, or any combination thereof.

7. The coronary perfusion device of claim 1, wherein the trough is an annular trough or a partial annular trough.

8. The coronary perfusion device of claim 1, wherein the coronary perfusion device is radially expandable and compressible.

9. The coronary perfusion device of claim 1, further comprising an annular frame, and wherein the trough is disposed at least partially within the frame.

10. The coronary perfusion device of claim 9, further comprising a skirt extending around an outer surface of the frame.

11. A coronary perfusion device for implantation in an ascending aorta of a patient comprising:
an annular frame;
a trough at least partially disposed inside the frame, the trough comprising an inner wall, an outer wall, an end wall extending between the inner wall and the outer wall, a first opening, and a second opening;
a first conduit extending from the first opening;
a second conduit extending from the second opening; and
a skirt extending around an outer surface of the frame;
wherein the device is configured to be implanted in the ascending aorta such that the first conduit extends into a right coronary artery and the second conduit extends into a left coronary artery; and
wherein the trough is configured to receive retrograde blood and direct the retrograde blood flow through the first and second conduits into the right coronary artery and the second coronary artery, respectively.

12. The coronary perfusion device of claim 11, wherein the frame and the trough are radially expandable and compressible between a radially expanded state and a radially compressed state.

13. The coronary perfusion device of claim 11, wherein the skirt is configured to engage the native ascending aorta to form a seal between the coronary perfusion device and the native ascending aorta.

14. The coronary perfusion device of claim 11, wherein the trough comprises an open mouth defined between the inner wall and the outer wall for receiving retrograde blood.

15. The coronary perfusion device of claim 14, wherein the mouth is configured to close or partially close when blood flows through the ascending aorta in an antegrade direction.

16. The coronary perfusion device of claim 14, wherein the mouth is configured to open or further open when blood flows through the ascending aorta in a retrograde direction.

17. The coronary perfusion device of claim 16, wherein the trough has a tapered cross-sectional profile when blood flows through the ascending aorta in a retrograde direction.

18. The coronary perfusion device of claim 11, wherein the trough is annular or partially annular.

19. The coronary perfusion device of claim 11, wherein the first opening and the second opening are located in the end wall.

20. A method for implanting a coronary perfusion device, the coronary perfusion device including a first conduit, a second conduit, and a circumferential trough connecting the first conduit and the second conduit, comprising:
implanting the trough in the ascending aorta of a patient downstream of the right and left coronary arteries;
positioning the first conduit in the right coronary artery of the patient; and
positioning the second conduit in the left coronary artery of the patient.
